(19) United States
(12) Patent Application Publication
(10) Pub. No.: US 2004/0037794 A1
(43) Pub. Date: Feb. 26, 2004

(54) Cosmetic composition comprising a starch betainate and a detergent surfactant

(76) Inventor: Claude Dubief, Le Chesnay (FR)

Correspondence Address: D Douglas Price, Steptoe & Johnson, 1330 Connecticut Avenue NW, Washington, DC, 20036, US

(21) Appl. No.: 10/333,208

(22) PCT Filed: Jul. 12, 2001
(86) PCT No.: PCT/FR01/02266

(51) Int. Cl. A61K 7/06

(52) U.S. Cl. 424/70.13

(57) ABSTRACT

The invention concerns a cosmetic treatment composition comprising, in a cosmetically acceptable aqueous medium, at least a starch betainate of formula (I), wherein: St represents a polymeric starch structure, and at least a detergent surfactant, the total amount of detergent being higher than 3 wt. % relative to the composition total weight. The invention also concerns a cosmetic treatment method for keratinous matter and use for washing skin and hair.

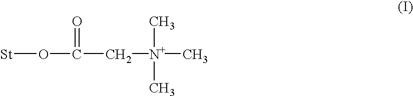

The present invention relates to a composition for the cosmetic treatment of keratinous materials, in particular the skin and the hair, comprising, in a cosmetically acceptable aqueous medium, at least one starch betainate and at least one detergent surfactant, to a method for the cosmetic treatment of keratinous materials and to a use for washing the skin or the hair.

The hair is generally damaged and embrittled by the action of external atmospheric agents such as light and adverse weather conditions, and by mechanical or chemical treatments such as brushing, combing, bleaching, permanent waving and/or dyeing. The result is that the hair is often difficult to disentangle or to style, and the hair, even when abundant, retains with difficulty a style of good appearance because of the fact that the hair lacks liveliness and vitality.

Thus, to remedy the above, it is now customary to apply to the hair conditioning agents which facilitate disentanglement and combing of wet hair, providing good retention of the hairstyle and imparting thereto, after drying, body, fullness and elasticity. For example, it is possible to use cationic starches which facilitate the disentanglement of wet hair, which improve the softness thereof and which provide it, once dry, with good properties of liveliness, hold and volume with no greasy feel or appearance.

However, repeated applications of these conditioning agents result in a rough feel of the hair and sometimes a lack of shine.

The applicant has discovered, surprisingly, that these undesirable effects can be avoided using at least one starch betainate combined with at least one detergent surfactant.

These starch betainates are novel polymers described in patent application WO 00/15669 from the company Raisio Chemicals OY and in the article “Preparation of starch betainate: a novel cationic starch derivative”. They are known only in the field of paper manufacture and have never been used in cosmetics.

These novel starch betainates are prepared from natural products and therefore exhibit good biodegradability. Combined with surfactants, they make it possible to obtain improved disentanglement and softness compared with the cationic starch and surfactant combinations of the prior art.

The subject of the present invention is therefore a composition for the cosmetic treatment of keratinous materials, and in particular of the skin and the hair, comprising, in a cosmetically acceptable aqueous medium, at least one starch betainate and at least one detergent surfactant.

Another subject of the invention consists in a method for the cosmetic treatment of keratinous materials using the composition according to the invention.

The subject of the invention is also a use of the composition according to the invention for washing the skin or the hair.

Other subjects, characteristics, features and advantages of the invention will appear even more clearly on reading the description and the various examples which follow.

According to the invention, the composition for the cosmetic treatment of keratinous materials comprises, in a cosmetically acceptable aqueous medium, at least one starch betainate corresponding to the following formula:

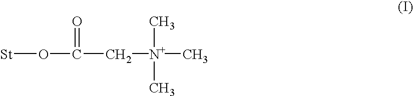

in which St represents a starch polymer structure, and at least one detergent surfactant, the total quantity of detergent surfactant(s) being greater than or equal to 3% by weight relative to the total weight of the composition.

The expression cosmetically acceptable aqueous medium is understood to mean a medium compatible with all keratinous materials such as the skin, the hair, the nails, the eyelashes and eyebrows, the lips and any other area of the body and of the face, but also having a pleasant odor, appearance and feel.

The starch molecules may be derived from any plant sources of starch such as, in particular, corn, potato, oats, rice, tapioca, sorghum, barley or wheat. Potato starch is preferred.

The starch betainates which can be used according to the present invention, and their preparation, and described in particular in application WO 00/15669 and in the article “Preparation of starch betainate: a novel cationic starch derivative”. They are prepared from natural products, namely a starch as described above and betaine.

The starch betainates may be used at a concentration of between 0.01 and 20% by weight, preferably between 0.05 and 10 % by weight, even better between 0.1 and 5% by weight relative to the total weight of the composition.

The detergent surfactants may be anionic, nonionic, or amphoteric surfactants or a mixture thereof.

As anionic surfactants which can be used, alone or as mixtures, in the context of the present invention, there may be mentioned especially salts, in particular alkali metal salts such as sodium salts, ammonium salts, amine salts, aminoalcohol salts or salts of alkaline earth metals, for example magnesium, of the following compounds: alkyl sulfates, alkyl ether sulfates, alkyl amidoether sulfates, alkyl aryl polyether sulfates, monoglyceride sulfates; alkyl sulfonates, alkyl phosphates, alkyl amide sulfonates, alkyl aryl sulfonates, [symbol] -olefin sulfonates, paraffin sulfonates; alkyl sulfosuccinates, alkyl ether sulfo-succinates, alkyl amide sulfosuccinates; alkyl sulfo-acetates; acyl sarcosinates; and acyl glutamates, the alkyl or acyl groups of all these compounds comprising from 6 to 24 carbon atoms and the aryl group preferably denoting a phenyl or benzyl group. It is also possible to use the esters of a C₆-C₂₄ alkyl and polyglycoside carboxylic acids such as alkyl glucoside citrates, alkyl polyglycoside tartrates and alkyl polyglycoside sulfosuccinates; alkyl sulfosuccinamates, acyl isethionates and N-acyltaurates, the alkyl or acyl group of all these compounds comprising from 12 to 20 carbon atoms. Among the anionic surfactants which can also be used, there may also be mentioned acyl lactylates in which the acyl group comprises from 8 to 20 carbon atoms.

In addition, there may also be mentioned alkyl D-galactoside uronic acids and their salts as well as polyoxyalkylenated (C₆-C₂₄)alkyl ether carboxylic acids, polyoxyalkylenated (C₆-C₂₄)alkyl (C₆-C₂₄)aryl ether carboxylic acids, polyoxyalkylenated (C₆-C₂₄)alkyl amidoether carboxylic acids and their salts, in particular those comprising from 2 to 50 ethylene oxide groups, and mixtures thereof.

Among the anionic surfactants cited above, the use of the salts of alkyl sulfates, alkyl ether sulfates and alkyl ether carboxylates, and mixtures thereof, is preferred according to the invention.

The nonionic surfactants are also compounds which are well known per se (see in particular in this regard “Handbook of Surfactants” by M. R. PORTER, published by Blackie & Son (Glasgow and London), 1991, pp. 116-178). Thus, they may be chosen in particular from alcohols, alpha-diols, (C₁-C₂₀)alkylphenols or polyethoxylated, polypropoxylated or polyglycerolated fatty acids, having a fatty chain comprising, for example, from 8 to 18 carbon atoms, it being possible for the number of ethylene oxide or propylene oxide groups to range in particular from 2 to 50 and it being possible for the number of glycerol groups to range in particular from 2 to 30. There may also be mentioned copolymers of ethylene and propylene oxides, condensates of ethylene and propylene oxides with fatty alcohols; polyethoxylated fatty amides preferably having from 2 to 30 mol of ethylene oxide, polyglycerolated fatty amides comprising on average 1 to 5 glycerol groups and in particular 1.5 to 4; polyethoxylated fatty amines preferably having 2 to 30 mol of ethylene oxide; ethoxylated fatty acid esters of sorbitan having from 2 to 30 mol of ethylene oxide; fatty acid esters of sucrose, fatty acid esters of polyethylene glycol, (C₆-C₂₄)alkyl polyglycosides, N-(C₆-C₂₄)alkylglucamine derivatives, amine oxides such as (C₀₀-C₁₄)alkylamine oxides or N-(C₁₀-C₁₄)acylaminopropylmorpholine oxides; and mixtures thereof.

Among the nonionic surfactants cited above, (C₆-C₂₄)-alkyl polyglycosides are preferably used.

The amphoteric surfactants which are suitable in the present invention may be in particular secondary or tertiary aliphatic amine derivatives in which the aliphatic group is a linear or branched chain comprising from 8 to 22 carbon atoms and containing at least one water-solubilizing anionic group such as, for example, a carboxylate, sulfonate, sulfate, phosphate or phosphonate group; there may also be mentioned (C₈-C₂₀)alkylbetaines, sulfobetaines, (C₈-C₂₀)alkylamido-(C₆-C₈)alkylbetaines or (C₈-C₂₀)alkylamido(C₆-C₈)alkylsulfobetaines; and mixtures thereof.

Among the amine derivatives, there may be mentioned the products marketed under the name MIRANOL®, as described in U.S. Pat. Nos. 2,528,378 and 2,781,354 and classified in the CTFA dictionary, 3rd edition, 1982, under the names Amphocarboxyglycinate and Amphocarboxypropionate having the respective structures (2) and (3):

R₂—CONHCH₂CH₂—N⁺(R₃)(R₄)(CH₂COO⁻) (2)

in which:

| | |
|---|---|
| R₂ | represents an alkyl group derived from an acid |
| | R₂-COOH present in hydrolyzed copra oil, a heptyl, |
| | nonyl or undecyl group, |
| R₃ | represents a beta-hydroxyethyl group, and |
| R₄ | represents a carboxymethyl group; |

and

R_{2′}—CONHCH₂CH₂—N(B)(C) (3)

in which:

| | |
|---|---|
| B | represents —CH₂CH₂OX′, |
| C | represents —(CH₂)₂—Y′, with z = 1 or 2, |
| X′ | represents the group —CH₂CH₂—COOH or a hydrogen |
| | atom, |
| Y′ | represents —COOH or the group —CH₂—CHOH—SO₃H, |
| R₂′ | represents an alkyl group of an acid R_{g}-COOH |
| | present in copra oil or in hydrolyzed linseed oil, |
| | an alkyl group, in particular a C₁₇ alkyl group and |
| | its iso form, an unsaturated C₁₇ group. |

These compounds are classified in the CTFA dictionary, 5th edition, 1993, under the names disodium cocoamphodiacetate, disodium lauroamphodiacetate, disodium caprylamphodiacetate, disodium capryloamphodiacetate, disodium cocoamphodipropionate, disodium lauroamphodipropionate, disodium caprylamphodipropionate, disodium capryloamphodipropionate, lauroamphodipropionic acid, cocoamphodipropionic acid.

By way of example, there may be mentioned the cocoamphodiacetate marketed under the trade name MIRANOL® C2M Concentrate by the company RHODIA.

Among the amphoteric surfactants, (C₈-C₂₀)alkylbetaines, (C₈-C₂₀)alkylamido(C₆-C₈) alkylbetaines, alkylamphodiacetates and mixtures thereof are preferably used.

The detergent surfactants described above may be used alone or as mixtures and their total quantity is greater than or equal to 3% by weight, preferably between 3% and 30% by weight, or even better between 3% and 25% by weight and at best between 3% and 20% by weight relative to the total weight of the composition.

The cosmetically acceptable aqueous medium may consist solely of water or of a mixture of water and of one cosmetically acceptable solvent such as a C₁-C₄ lower alcohol, such as ethanol, isopropanol, tert-butanol, n-butanol; alkylene glycols such as propylene glycol, glycol ethers.

The composition for cosmetic treatment of the invention may comprise, in addition, at least one cationic polymer different from the above starch betainate.

The expression cationic polymer is understood to mean any polymer containing cationic groups and/or groups which can be ionized into cationic groups.

The cationic polymers which can be used in accordance with the present invention may be chosen from all those already known per se to improve the cosmetic properties of hair treated with detergent compositions, namely in particular those described in patent application EP-A-0 337 354 and in French patent applications FR-A-2 270 846, 2 383 660, 2 598 611, 2 470 596 and 2 519 863.

The preferred cationic polymers are chosen from those which contain units comprising primary, secondary, tertiary and/or quaternary amine groups which may either form part of the principal polymer chain, or which may be carried by a side substituent directly linked thereto.

The cationic polymers used generally have a number-average molecular mass of between 500 and 5·10⁶ approximately, and preferably of between 103 and 3·10⁶ approximately.

Among the cationic polymers, there may be mentioned more particularly the polymers of the polyamine, polyaminoamide and poly(quaternary ammonium) type. They are known products.

The polymers of the polyamine, polyaminoamide and poly(quaternary ammonium) type which can be used in the composition of the present invention are those described in French Patent No. 2 505 348 or 2 542 997. Among these polymers, there may be mentioned:

(1) the homopolymers or copolymers derived from acrylic or methacrylic esters or amides and comprising at least one of the units of the following formulae:

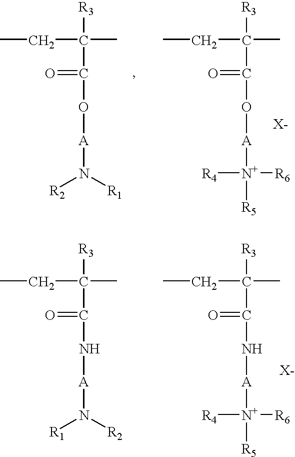

in which:

R₃, which are identical or different, denote a hydrogen atom or a CH₃ group;

A, which are identical or different, represent a linear or branched alkyl group comprising from 1 to 6 carbon atoms, preferably 2 or 3 carbon atoms, or a hydroxyalkyl group comprising from 1 to 4 carbon atoms;

R₄, R₅, R₆, which are identical or different, represent an alkyl group having from 1 to 18 carbon atoms or a benzyl group, and preferably an alkyl group having from 1 to 6 carbon atoms;

R₁ and R₂, which are identical or different, represent a hydrogen atom or an alkyl group having from 1 to 6 carbon atoms and preferably a methyl or ethyl group;

X denotes an anion derived from an inorganic or organic acid such as a methosulfate anion or a halide such as chloride or bromide.

The copolymers of the family (1) may contain, in addition, one or more units derived from comonomers which may be chosen from the family of acrylamides, methacrylamides, diacetone acrylamides, acrylamides and methacrylamides substituted on the nitrogen atom with lower (C₁-C₄)alkyl groups, groups derived from acrylic or methacrylic acids or esters thereof, vinyllactams such as vinylpyrrolidone or vinylcaprolactam, vinyl esters.

Thus, among these copolymers of the family (1), there may be mentioned:

the copolymers of acrylamide and dimethylaminoethyl methacrylate quaternized with dimethyl sulfate or with a dimethyl halide such as that sold under the name HERCOFLOC® by the company HERCULES,

the copolymers of acrylamide and methacryloyloxy-ethyltrimethylammonium chloride described, for example, in Patent Application EP-A-080976 and sold under the name BINAQUAT® P 100 by the company CIBA GEIGY,

the copolymer of acrylamide and methacryloyl-oxyethyltrimethylammonium methosulfate sold under the name RETEN® by the company HERCULES,

the vinylpyrrolidone/dialkylaminoalkyl acrylate or methacrylate copolymers, quaternized or otherwise, such as the products sold under the name “GAFQUAT®” by the company ISP such as for example “GAFQUAT® 734” or “GAFQUAT® 755”, or alternatively the products called “COPOLYMER 845, 958 and 937”. These polymers are described in detail in French Patents 2,077,143 and 2,393,573,

the dimethylaminoethyl methacrylate/vinylcaprolactam/vinylpyrrolidone terpolymers such as the product sold under the name GAFFIX® VC 713 by the company ISP,

the vinylpyrrolidone/methacrylamidopropyldimethylamine copolymers marketed in particular under the name STYLEZE® CC 10 by ISP, and

the quaternized vinylpyrrolidone/dimethylaminopropylmethacrylamide copolymers such as the product sold under the name “GAFQUAT® HS 100” by the company ISP.

(2) The cellulose ether derivatives comprising quaternary ammonium groups, described in French Patent 1,492,597, and in particular the polymers marketed under the names “JR” (JR 400, JR 125, JR 30M) or “LR” (LR 400, LR 30M) by the company Union Carbide Corporation. These polymers are also defined in the CTFA dictionary as hydroxyethyl cellulose quaternary ammoniums which have reacted with an epoxide substituted by a trimethylammonium group.

(3) Cationic cellulose derivatives such as cellulose copolymers or cellulose derivatives grafted with a quaternary ammonium water-soluble monomer, and described especially in U.S. Pat. No. 4,131,576, such as hydroxyalkyl celluloses like hydroxymethyl, hydroxyethyl or hydroxypropyl celluloses grafted especially with a methacryloylethyltrimethylammonium, methacrylamidopropyltrimethylammonium or dimethyldiallylammonium salt.

The commercialized products corresponding to this definition are more particularly the products sold under the name “Celquat® L 200” and “Celquate H 100” by the company National Starch.

(4) The cationic polysaccharides described more particularly in U.S. Pat. Nos. 3,589,578 and 4,031,307 such as guar gums containing cationic trialkylammonium groups.

Guar gums modified with a 2,3-epoxypropyltrimethylammonium salt (e.g. chloride) are for example used.

Such products are marketed in particular under the trade names JAGUAR® C13 S, JAGUAR® C 15, JAGUAR® C 17 or JAGUAR® C162 by the company MEYHALL.

(5) Polymers consisting of piperazinyl units and of alkylene or hydroxyalkylene divalent groups with straight or branched chains, optionally interrupted by oxygen, sulfur or nitrogen atoms or by aromatic or heterocyclic rings, as well as the oxidation and/or quaternization products of these polymers. Such polymers are described especially in French patents 2,162,025 and 2,280,361.

(6) Water-soluble polyaminoamides prepared in particular by polycondensation of an acid compound with a polyamine; these polyaminoamides may be crosslinked with an epihalohydrin, a diepoxide, a dianhydride, an unsaturated dianhydride, a diunsaturated derivative, a bishalohydrin, a bisazetidinium, a bishaloacyldiamine, an alkyl bishalide or else with an oligomer resulting from the reaction of a difunctional compound which is reactive toward a bishalohydrin, a bisazetidinium, a bishaloacyldiamine, an alkyl bishalide, an epihalohydrin, a diepoxide or a diunsaturated derivative; the crosslinking agent being employed in proportions ranging from 0.025 to 0.35 mol per amine group of the polyaminoamide; these polyaminoamides may be alkylated or, if they include one or more tertiary amine functional groups, quaternized. Such polymers are described especially in French Patents 2,252,840 and 2,368, 508.

(7) Polyaminoamide derivatives resulting from the condensation of polyalkylenepolyamines with polycarboxylic acids, followed by an alkylation with difunctional agents. There may be mentioned, for example, the adipic acid/dialkylaminohydroxyalkyl-dialkylenetriamine polymers in which the alkyl group contains from 1 to 4 carbon atoms and preferably denotes a methyl, ethyl or propyl group and the alkylene group contains from 1 to 4 carbon atoms, and preferably denotes the ethylene group. Such polymers are described especially in French Patent 1,583,363.

Among these derivatives there may be mentioned more particularly the adipic acid/dimethylaminohydroxypropyl/diethylenetriamine polymers sold under the name “Cartaretine® F, F4 or F8” by the company Sandoz.

(8) Polymers obtained by reaction of a polyalkylenepolyamine containing two primary amine groups and at least one secondary amine group with a dicarboxylic acid chosen from diglycolic acid and saturated aliphatic dicarboxylic acids containing from 3 to 8 carbon atoms. The molar ratio of the polyalkylenepolyamine to the dicarboxylic acid being between 0.8:1 and 1.4:1; the polyaminoamide resulting therefrom being made to react with epichlorohydrin in a molar ratio of epichlorohydrin relative to the secondary amine group of the polyaminoamide of between 0.5:1 and 1.8:1. Such polymers are described especially in American Patents 3,227,615 and 2,961,347.

Polymers of this type are marketed in particular under the name “Hercosett® 57” by the company Hercules Inc. or else under the name of “PD 170” or “Delsette® 101” by the company Hercules in the case of the copolymer of adipic acid/epoxypropyl-diethylene-triamine.

(9) Cyclopolymers of alkyldiallylamine or of dialkyldiallylammonium, such as the homopolymers or copolymers comprising, as main constituent of the chain, units corresponding to the formulae (Va) or (Vb):

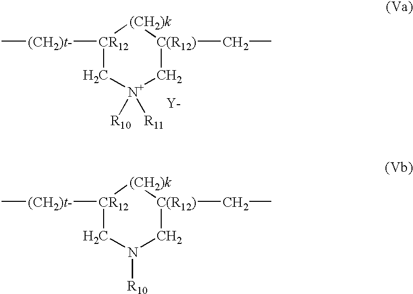

in which formulae k and t are equal to 0 or 1, the sum k+t being equal to 1; R₁₂ denotes a hydrogen atom or a methyl group; R₁₀ and R₁₁, independently of each other, denote an alkyl group containing from 1 to 22 carbon atoms, a hydroxyalkyl group in which the alkyl group preferably has 1 to 5 carbon atoms, or a lower (C₁-C₄)-amidoalkyl group, or R₁₀ and R₁₁ may denote, jointly with the nitrogen atom to which they are attached, heterocyclic groups such as piperidinyl or morpholinyl; Y⁻ is an anion such as bromide, chloride, acetate, borate, citrate, tartrate, bisulfate, bisulfite, sulfate or phosphate. These polymers are described especially in French Patent 2,080,759 and in its certificate of addition 2,190,406.

R₁₀ and R₁₁, independently of each other, preferably denote an alkyl group having from 1 to 4 carbon atoms.

Among the polymers defined above there may be mentioned more particularly the dimethyldiallylammonium chloride homopolymer sold under the name “MERQUAT® 100” by the company CALGON (and its homologues of low weight-average molecular masses) and the copolymers of diallyl-dimethylammonium chloride and acrylamide marketed under the name “MERQUAT® 550”.

(10) The quaternary diammonium polymer containing repeat units corresponding to the formula (VI):

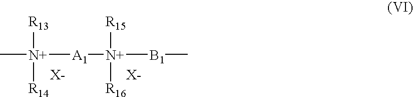

in which:

R₁₃, R₁₄, R₁₅ and R₁₆, which are identical or different, represent aliphatic, alicyclic or arylaliphatic groups containing from 1 to 20 carbon atoms or lower hydroxyalkyl aliphatic groups, or else R₁₃, R₁₄, R₁₅ and R₁₆, together or separately, form, with the nitrogen atoms to which they are attached, heterocyclic rings optionally containing a second heteroatom other than nitrogen, or else R₁₃, R₁₄, R₁₅ and R₁₆ denote a linear or branched C₁-C₆ alkyl group substituted by a nitrile, ester, acyl, amide or —CO—O—R₁₇-D or —CO—NH—R₁₇-D group where R₁₇ is an alkylene group and D a quaternary ammonium group;

A₁ and B₁ represent polymethylene groups containing from 2 to 20 carbon atoms which may be linear or branched, saturated or unsaturated and which may contain, bonded to or inserted into the main chain, one or more aromatic rings, or one or more oxygen or sulfur atoms or sulfoxide, sulfone, disulfide, amino, alkylamino, hydroxyl, quaternary ammonium, ureido, amide or ester groups, and

X⁻ denotes an anion derived from an inorganic or organic acid;

A₁, R₁₃ and R₁₅, with the two nitrogen atoms to which they are attached, may form a piperazine ring; in addition if A₁ denotes a saturated or unsaturated, linear or branched alkylene or hydroxyalkylene group, B₁ may also denote a group:

—(CH₂)ₙ—CO-D-OC—(CH₂)ₙ—

in which D denotes:

a) a glycol residue of formula —O-Z-O—, where Z denotes a linear or branched hydrocarbon group or a group corresponding to one of the following formulae:

—(CH₂—CH₂—O)ₓ—CH₂—CH₂—

—[CH₂—CH(CH₃)—O]_{y}—CH₂—CH(CH₃)—

where x and y denote an integer from 1 to 4, representing a defined and unique degree of polymerization or any number from 1 to 4 representing a mean degree of polymerization;

b) a disecondary diamine residue such as a piperazine derivative;

c) a diprimary diamine residue of formula —NH—Y—NH—, where Y denotes a linear or branched hydrocarbon group or else the divalent group —CH₂—CH₂—S—S—CH₂—CH₂—;

d) a ureylene group of formula —NH—CO—NH—.

X⁻ is preferably an anion such as chloride or bromide.

These polymers have a number-average molecular mass which is generally between 1000 and 100,000.

Polymers of this type are described especially in French Patents 2,320,330, 2,270,846, 2,316,271, 2,336,434 and 2,413,907 and U.S. Pat. Nos. 2,273,780, 2,375,853, 2,388,614, 2,454,547, 3,206,462, 2,261,002, 2,271,378, 3,874,870, 4,001,432, 3,929,990, 3,966,904, 4,005,193, 4,025,617, 4,025,627, 4,025,653, 4,026,945 and 4,027,020.

It is possible to use more particularly the polymers which consist of repeat units corresponding to the formula:

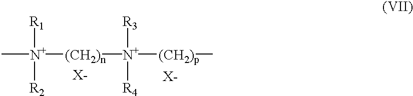

in which R₁, R₂, R₃ and R₄, which are identical or different, denote an alkyl or hydroxyalkyl group having from 1 to 4 carbon atoms approximately, n and p are integers varying from 2 to 20 approximately and X⁻ is an anion derived from an inorganic or organic acid.

An especially preferred compound of formula (VII) is that for which R₁, R₂, R₃ and R₄ represent a methyl group and n=3, p=6 and X=Cl, called hexadimethrine chloride (CTFA).

(11) Poly(quaternary ammonium) polymers consisting of units of formula (VIII):

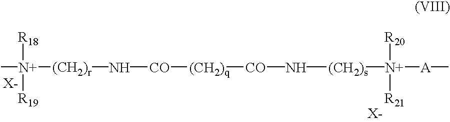

in which:

R₁₈, R₁₉, R₂₀ and R₂₁, which are identical or different, represent a hydrogen atom or a methyl, ethyl, propyl, [symbol] -hydroxyethyl, [symbol] -hydroxypropyl or —CH₂CH₂(OCH₂CH₂)ₚOH group, where p is equal to 0 or to an integer between 1 and 6, provided that R₁₈, R₁₉, R₂₀ and R₂₁ do not simultaneously represent a hydrogen atom,

r and s, which are identical or different, are integers between 1 and 6,

q is equal to 0 or to an integer between 1 and 34,

X⁻ denotes an anion such as a halide,

A denotes a radical of a dihalide or preferably represents —CH₂—CH₂—O—CH₂—CH₂—.

Such compounds are described especially in Patent Application EP-A-122 324.

Among these there may be mentioned, for example, the products “Mirapol® A 15”, “Mirapol® AD1”, “Mirapol® AZ1” and “Mirapole 175”, sold by the company Miranol.

(12) Quaternary vinylpyrrolidone and vinylimidazole polymers such as, for example, the products marketed under the names Luviquat® FC 905, FC 550 and FC 370 by the company B.A.S.F.

(13) Polyamines like Polyquart® H sold by HENKEL, referred to under the name of “POLYETHYLENE GLYCOL (15) TALLOW POLYAMINE” in the CTFA dictionary.

(14) The crosslinked polymers of methacryloyloxy(C₁-C₄ alkyl)tri(C₁-C₄ alkyl)ammonium salts such as the polymers obtained by homopolymerization of dimethylaminoethyl methacrylate quaternized with methyl chloride, or by copolymerization of acrylamide with dimethylaminoethyl methacrylate quaternized with methyl chloride, the homo- or copolymerization being followed by crosslinking with a compound containing olefinic unsaturation, in particular methylenebisacrylamide. More particularly, it is possible to employ a crosslinked acrylamide/methacryloyloxyethyltrimethylammonium chloride copolymer (20/80 by weight) in the form of a dispersion containing 50% by weight of said copolymer in mineral oil. This dispersion is marketed under the name of “SALCARE® SC 92” by the company ALLIED COLLOIDS. It is also possible to employ a crosslinked methacryloyloxyethyltrimethylammonium chloride homopolymer containing approximately 50% by weight of the homopolymer in mineral oil or in a liquid ester. These dispersions are marketed under the names of SALCARE® SC 95 and SALCARE® SC 96 by the company ALLIED COLLOIDS.

Other cationic polymers that may be employed within the scope of the invention are cationic proteins or hydrolyzates of cationic proteins, polyalkyleneimines, in particular polyethyleneimines, polymers containing vinylpyridine or vinylpyridinium units, condensates of polyamines and of epichlorohydrin, quaternary polyureylenes and chitin derivatives.

Among all the cationic polymers capable of being used within the scope of the present invention, it is preferable to use quaternary cellulose ether derivatives such as the products sold under the name “JR 400” by the company UNION CARBIDE CORPORATION, cationic cyclopolymers, in particular the homopolymers or copolymers of dimethyldiallylammonium chloride, sold under the names MERQUAT® 100, MERQUAT® 550 and MERQUAT® S by the company CALGON, cationic polysaccharides such as guar gums modified by a salt of 2,3-epoxypropyltrimethylammonium, quaternary polymers of vinylpyrrolidone and vinylimidazole, and mixtures thereof.

Among the cationic polymers mentioned above, which are suitable in the invention, there are preferably used quaternary cellulose ether derivatives, cationic cyclopolymers, quaternary polymers of vinylpyrrolidone and of vinylimidazole, and mixtures thereof.

The compositions according to the invention may also comprise one or more amphoteric polymers. Among them, there may be mentioned most particularly acrylic acid/dialkyldiallylammonium chloride copolymers such as MERQUAT® 100 sold by the company CALGON.

According to the invention, the cationic polymer(s) may represent from 0.001% to 10% by weight, preferably from 0.005% to 5% by weight, and more particularly from 0.01 to 3% by weight relative to the total weight of the final composition.

The pH of the compositions of the invention is between 4 and 8, preferably between 5 and 7.

The compositions according to the invention may also contain additives such as associative or non-associative, anionic, amphoteric, zwitterionic, non-ionic or cationic, natural or synthetic polymeric thickeners, nonpolymeric thickeners such as acids or electrolytes, pearlescent agents, opacifying agents, organic solvents, perfumes, inorganic, vegetable and/or synthetic oils, fatty acid esters, colorants, branched or unbranched, cyclic or acyclic, organomodified or nonorganomodified, volatile or nonvolatile silicones, inorganic or organic particles, preservatives, pH-stabilizing agents.

Persons skilled in the art will be careful to choose the optional additives and their quantity so that they do not damage the properties of the compositions of the present invention.

These additives are present in the composition according to the invention in a quantity ranging from 0 to 20% by weight relative to the total weight of the composition.

The compositions may be provided in the form of fluid or thickened liquids, gels, creams, foams, W/O emulsions, OW emulsions or multiple emulsions.

They may be used, for example, as shampoos, rinse-out or leave-in treatments, deep treatment masks, lotions or creams for treating the scalp and the skin, make-up removers.

The present invention also relates to a method for the cosmetic treatment of keratinous materials which consists in applying to the keratinous materials an effective quantity of a composition as described above, and in optionally rinsing after an optional exposure time.

The following examples illustrate the present invention and should not be considered in any manner as limiting the invention.

### EXAMPLE 1

A shampoo according to the invention was prepared from the following ingredients:

| | | | |
|---|---|---|---|
| Sodium lauryl ether sulfate (2.2 mol of | | 17 | g |
| ethylene oxide) | | | |
| Cocoylbetaine | | 2.5 | g |
| Potato starch betainate having a degree | | 0.05 | g |
| of substitution of 0.8 | | | |
| Water, perfume, preservative | qs | 100 | g |
| pH adjusted to 6 (HCl/NaOH) | | | |

After rinsing, the hair is soft, smooth and easy to disentangle.

### EXAMPLE 2

A shampoo according to the invention was prepared from the following ingredients:

| | | | |
|---|---|---|---|
| Sodium lauryl ether sulfate (2.2 mol of | | 6 | g |
| ethylene oxide) | | | |
| Sodium N-cocoylamidoethyl-N-carboxy- | | 3.5 | g |
| methylglycinate (Miranol ® CLM Concentrate | | | |
| NP from RHODIA) | | | |
| Potato starch betainate having a degree | | 0.8 | g |
| of substitution of 0.8 | | | |
| Water, perfume, preservative | qs | 100 | g |
| pH adjusted to 6 (HCl/NaOH) | | | |

After rinsing, the hair is soft, smooth and easy to disentangle.

### EXAMPLE 3

A shampoo according to the invention was prepared from the following ingredients:

| | | | |
|---|---|---|---|
| Sodium lauryl ether sulfate (2.2 mol of | | 17 | g |
| ethylene oxide) | | | |
| Cocoylbetaine | | 2.5 | g |
| Sodium ketostearyl sulfate | | 0.75 | g |
| Mixture of cetyl alcohol and of 1-(hexa- | | 2.5 | g |
| decyloxy)-2-octadecanol | | | |
| Copra acid monoisopropanolamide | | 0.6 | g |
| Polydimethylsiloxane (300 000 mm²/s) | | 2.7 | g |
| (DC200 fluid 300 000 from DOW CORNING) | | | |
| Potato starch betainate having a degree | | 1 | g |
| of substitution of 0.8 | | | |
| Water, perfume, preservative | qs | 100 | g |

After rinsing, the hair is soft, smooth and easy to disentangle.

1. A composition for the cosmetic treatment of keratinous materials, characterized in that it comprises, in a cosmetically acceptable aqueous medium, at least one starch betainate corresponding to the following formula: 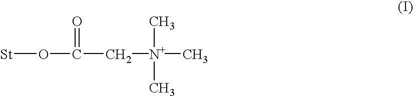
in which St represents a starch polymer structure, and at least one detergent surfactant, the total quantity of detergent surfactant(s) being greater than or equal to 3% by weight relative to the total weight of the composition:

2. The composition as claimed in claim 1, characterized in that the starch is chosen from corn, potato, oat, rice, tapioca, sorghum, barley or wheat starches.

3. The composition as claimed in claim 1, characterized in that the detergent surfactants are chosen from anionic, nonionic, or amphoteric surfactants and mixtures thereof.

4. The composition as claimed in claim 3, characterized in that the anionic surfactants are chosen from the salts of the following compounds: alkyl sulfates, alkyl ether sulfates, alkyl amidoether sulfates, alkyl aryl polyether sulfates, monoglyceride sulfates; alkyl sulfonates, alkyl phosphates, alkyl amide sulfonates, alkyl aryl sulfonates, [symbol] -olefin sulfonates, paraffin sulfonates; alkyl sulfosuccinates, alkyl ether sulfosuccinates, alkyl amide sulfosuccinates; alkyl sulfoacetates; acyl sarcosinates; and acyl glutamates; the alkyl or acyl groups of these compounds comprising from 6 to 24 carbon atoms and the aryl group preferably denoting a phenyl or benzyl group; the esters of a C₆-C₂₄ alkyl and polyglycoside carboxylic acids; alkyl sulfosuccinamates, acyl isethionates and N-acyltaurates, the alkyl or acyl group comprising from 12 to 20 carbon atoms; acyl lactylates in which the acyl group comprises from 8 to 20 carbon atoms; alkyl D-galactoside uronic acids and their salts, polyoxyalkylenated (C₆-C₂₄)alkyl ether carboxylic acids, polyoxyalkylenated (C₆-C₂₄)alkyl (C₆-C₂₄)aryl ether carboxylic acids, polyoxyalkylenated (C₆-C₂₄)alkyl amidoether carboxylic acids and their salts; and mixtures thereof.

5. The composition as claimed in claim 4, characterized in that the anionic surfactants are chosen from the salts of alkyl sulfates, alkyl ether sulfates and alkyl ether carboxylates, and mixtures thereof.

6. The composition as claimed in claim 3, characterized in that the nonionic surfactants are chosen from alcohols, alpha-diols, (C₁-C₂₀)alkylphenols or polyethoxylated, polypropoxylated or polyglycerolated C₈-C₁₈ fatty acids, the number of ethylene oxide or propylene oxide groups ranging from 2 to 50 and the number of glycerol groups ranging from 2 to 30, copolymers of ethylene and propylene oxides, condensates of ethylene and propylene oxides with fatty alcohols; polyethoxylated fatty amides having from 2 to 30 mol of ethylene oxide, polyglycerolated fatty amides comprising on average 1 to 5 glycerol groups; polyethoxylated fatty amines having from 2 to 30 mol of ethylene oxide; ethoxylated fatty acid esters of sorbitan having from 2 to 30 mol of ethylene oxide; fatty acid esters of sucrose, fatty acid esters of polyethylene glycol, (C₆-C₂₄)alkyl polyglycosides, N-(C₆-C₂₄)alkylglucamine derivatives, amine oxides; and mixtures thereof.

7. The composition as claimed in claim 3, characterized in that the amphoteric surfactants are chosen from secondary or tertiary aliphatic amine derivatives in which the aliphatic group is a linear or branched chain comprising from 8 to 22 carbon atoms and containing at least one water-solubilizing anionic group such as a carboxylate, sulfonate, sulfate, phosphate or phosphonate group; (C₈-C₂₀)alkylbetaines, sulfobetaines, (C₈-C₂₀) alkylamido (C₆-C₈) alkylbetaines or (C₈-C₂₀)-alkylamido(C₆-C₈)alkylsulfobetaines; and mixtures thereof.

8. The composition as claimed in any one of the preceding claims, characterized in that the total quantity of detergent surfactant(s) is between 3% and 30% by weight.

9. The composition as claimed in any one of the preceding claims, characterized in that the starch betainate is present in a quantity of between 0.01 and 20% by weight.

10. The composition as claimed in claim 9, characterized in that the starch betainate is present in a quantity of between 0.05 and 10% by weight.

11. The composition as claimed in claim 1, characterized in that the cosmetically acceptable aqueous medium consists solely of water or of a mixture of water and of a cosmetically acceptable solvent.

12. The composition as claimed in claim 11, characterized in that the cosmetically acceptable solvent is chosen from C₁-C₄ lower alcohols, such as ethanol, isopropanol, tert-butanol, n-butanol; alkylene glycols such as propylene glycol, glycol ethers.

13. The composition as claimed in any one of the preceding claims, characterized in that it comprises, in addition, another cationic polymer.

14. The composition as claimed in claim 13, characterized in that the cationic polymer is chosen from quaternary cellulose ether derivatives, cationic cyclopolymers, quaternary polymers of vinylpyrrolidone and of vinylimidazole, and mixtures thereof.

15. The composition as claimed in any one of the preceding claims, characterized in that it comprises, in addition, additives such as associative or nonassociative, anionic, amphoteric, zwitterionic, nonionic or cationic, natural or synthetic polymeric thickeners, nonpolymeric thickeners such as acids or electrolytes, pearlescent agents, opacifying agents, organic solvents, perfumes, inorganic, vegetable and/or synthetic oils, fatty acid esters, colorants, branched or unbranched, cyclic or acyclic, organomodified or nonorganomodified, volatile or nonvolatile silicones, inorganic or organic particles, preservatives, pH-stabilizing agents.

16. A method for the cosmetic treatment of keratinous materials, characterized in that a composition as claimed in any one of the preceding claims is applied to the keratinous materials, and in that rinsing is optionally carried out after an optional exposure time.

17. The use of a composition for cosmetic treatment as claimed in any one of claims 1 to 15, for washing the skin or the hair.
